(19) United States
(12) Patent Application Publication
(10) Pub. No.: US 2022/0168665 A1
(43) Pub. Date: Jun. 2, 2022

(54) PROCESS FOR MAKING DRUG CRYSTALS OF DESIRED SIZE DISTRIBUTION AND MORPHOLOGY

(71) Applicant: EUPRAXIA PHARMACEUTICALS INC., Victoria (CA)

(72) Inventors: Azadeh GOUDARZI, Victoria (CA); Troy LOSS, Victoria (CA); Michael GAINES, Victoria (CA); Gina STETSKO, Victoria (CA); Amanda MALONE, Victoria (CA)

(21) Appl. No.: 17/594,208

(22) PCT Filed: Apr. 10, 2020
(86) PCT No.: PCT/US20/27797

§ 371 Date: Oct. 6, 2021

Related U.S. Application Data
(60) Provisional application No. 62/832,179, filed on Apr. 10, 2019.

(51) Int. Cl. B01D 9/00; A61K 9/50; A61K 31/56; B07B 13/04

(52) U.S. Cl. CPC B01D 9/0059; A61K 9/5026; A61K 9/5031; B07B 13/04; A61K 31/56; B01D 9/0045; A61K 9/5089

(57) ABSTRACT

Provided herein includes a process for forming drug crystals of narrow size distribution and desire dimensions and morphology, the process includes a recrystallization step followed by a resizing step.

### BACKGROUND

### Technical Field

This disclosure relates to a process for making drug crystals of certain particle size distributions and morphology, which are particularly suitable for being coated to provide sustained released formulations.

### Description of the Related Art

Drug crystals can be fully coated or encapsulated in a thin polymer membrane and slowly released in a sustained manner. U.S. Pat. No. 9,987,233 discloses polyvinyl alcohol-coated fluticasone propionate crystals, which can be injected into joints or other body compartments, whereby fluticasone propionate is released locally over a long period of time while producing no clinically significant HPA axis suppression due to low systemic exposure.

Fluid bed equipment is commonly used for coating or encapsulating discrete solid particles, including drug crystals. Such equipment utilizes differential air flow to float solid particles while a nozzle sprays an atomized coating material (e.g., droplets of a polymer solution) to coat the solid particles individually.

Fluid bed equipment functions most optimally when the drug crystals to be coated have a controlled, reproducible and uniform particle size distribution. Non-uniform distributions of particle size affect the optimization process for the floating parameters. For instance, when coating parameters are optimized for smaller particles to float, the larger particles remain immobile. Conversely, when parameters are optimized for larger particles, the smaller particles may be shattered by the high impact collision to the side walls and/or become agglomerated with uneven coating coverage. Fine particles (“fines”), e.g., those that are less than 15% of the mean diameter, can cause additional issues by clogging the equipment filters and/or alter the quality of the coating by agglomeration. The shapes of the particles also impact coating quality because they affect the particles' buoyancy and ability to float.

Thus, there is a need in the art to reproducibly provide drug crystals with desired and controllable size distribution and morphology.

### BRIEF SUMMARY

Provided here are a plurality or collection of drug crystals having desired size, shape and size distribution suitable for homogeneous coating via the fluid bed method. Also disclosed are method for producing the same.

In a particular embodiment, the drug crystals are fluticasone propionate (FP) crystals with cube-like morphology (i.e., similar size in all dimensions) and narrow particle size distribution in the range of 50-250 μm.

It should be noted that the method and process described herein are not limited to fluticasone esters such as fluticasone propionate. Rather, other drug substances, especially poor soluble drugs (such as corticosteroids) that favor lengthwise crystal growth, can be recrystallized and resized according to one or more embodiments.

### BRIEF DESCRIPTION OF THE SEVERAL VIEWS OF THE DRAWINGS

FIG. 1 shows a flow chart of the process of preparing a population of sized drug crystals according to an embodiment.

FIG. 2A-2C show a raw, as-received drug material or active pharmaceutical ingredient (API) that has been recrystallized to thick, elongated columnar crystals before being sized to drug crystals of target dimensions.

FIG. 3 shows monoclinic crystal Form I of fluticasone propionate (FP).

FIG. 4 schematically shows the crystal growth mechanism for producing thick elongated crystals.

FIGS. 5-6 show the SEM (Scanning Electron Microscope) images of recrystallized FP crystals obtained under an isothermal condition in small and large scales, respectively.

FIGS. 7-8 show the SEM images of recrystallized FP crystals obtained under another isothermal condition in small and large scales, respectively.

FIG. 9 shows the X-Ray diffraction patterns of the raw drug material (commercial source) and the recrystallized FP crystals according to an embodiment.

FIG. 10 shows a bench top rotor/stator homogenizer.

FIG. 11 shows the SEM image of the sized (milling followed by sieving) drug crystals.

FIG. 12 shows the particle size distribution of the sized drug crystals according to an embodiment.

FIG. 13 shows a side-by-side comparison of the commercial source of the FP crystals and the sized drug crystals obtained according to an embodiment of the present disclosure.

### DETAILED DESCRIPTION

Described herein include a process for providing bulk crystals, of certain target size distribution and morphology. The process comprises (1) providing recrystallized columnar crystals having aspect ratios of large than 1 and less than 20, the aspect ratio of a given columnar crystal being the ratio of a longest dimension along a lengthwise axis relative to a shortest dimension of a transverse plane perpendicular to the lengthwise axis; and (2) sizing the recrystallized columnar crystals to provide a collection of sized crystals having certain target dimensions, wherein the sizing includes segmenting at least a portion of the recrystallized columnar crystals along the respective lengthwise axes while retaining the dimensions of the transverse plane perpendicular to the lengthwise axis.

Although the process is applicable to any crystals, it is particularly suited for generating bulk drug crystals, i.e., a collection of sized drug crystals of monodispersed size, shapes and mass distribution, making them particularly suited for being coated by a fluid bed type of coating equipment.

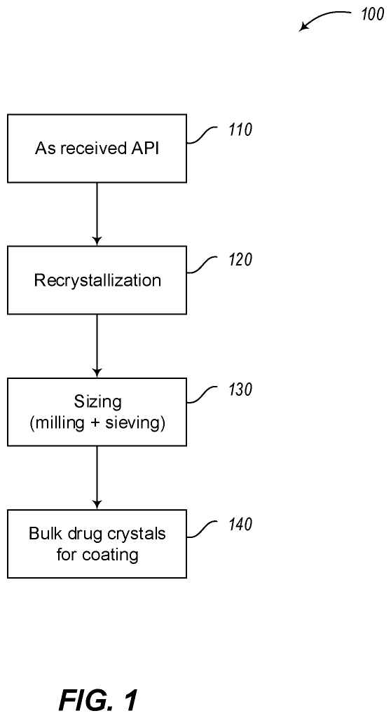

FIG. 1 shows schematically a process according to an embodiment of the present disclosure. The process (100) utilizes a raw drug substance such as an API from a commercial source (110), which are typically micronized crystals (e.g., less than 5 microns). The as-received API (110) is then recrystallized (120) to provide columnar crystals having aspect ratios in the range of larger than 1 and less than 20, the aspect ratio of a given crystal being the ratio of a longest dimension along a lengthwise axis relative to a shortest dimension of a transverse plane perpendicular to the lengthwise axis. The recrystallized columnar crystals are thereafter sized (130) to provide bulk drug crystals of target dimensions. The sizing step comprises milling by which at least a portion of the recrystallized columnar crystals are preferentially segmented along the lengthwise axis, i.e., the dimensions of the transverse plane perpendicular to the lengthwise axis are retained. The segmented crystals are sieved and those that meet certain target dimensions are collected. The resulting bulk drug crystals (140) comprise a collection of sized crystals having a narrow size distribution, as defined herein. In particular, the bulk drug crystals have substantially uniform shapes, sizes and mass distribution, and are suited for fluid bed coating.

In certain embodiments, a sized crystal is shortened from a recrystallized columnar crystal yet retains the dimensions of the transverse plane perpendicular to the lengthwise axis of the recrystallized columnar crystal. In certain embodiments, the sized crystals have aspect ratios in the range of 1-3. In more specific embodiments, the sized drug crystal are cube-shaped and have an aspect ratio of around 1. As used herein, “substantially the same” of “substantially uniform” refers to no more than 25%, or preferably no more than 15%, or preferably no more than 5% of differences.

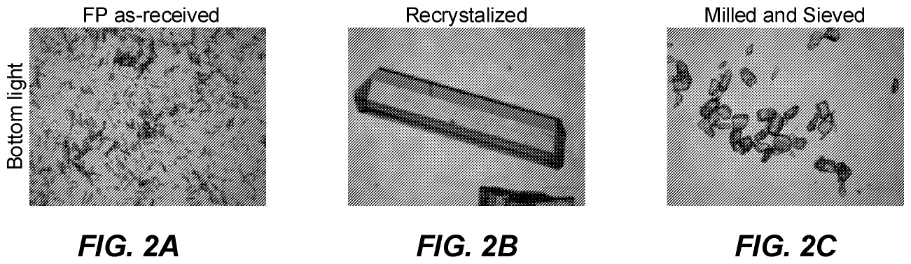

FIG. 2A-2C show a specific embodiment by which a raw drug material (e.g., commercially available crystalline fluticasone propionate, shown in FIG. 2A) were first recrystallized to large, columnar crystals (FIG. 2B), followed by sizing (e.g., shortening the recrystallized columnar crystals and sieving) into sized crystals of narrow size distribution, as defined herein (FIG. 2C).

To effectively carry out the sizing process, it is important that the recrystallized columnar crystals to be sized first meet certain target dimensions. In more specific embodiments, the target dimensions of the recrystallized columnar drug crystals meet one or more of the following criteria: (i) 90% of the total mass are no larger than 1200 microns (D₉₀); (ii) 50% of the total mass are less than 350±180 microns (D₅₀); (iii) no more than 10% of the total mass are less than 50 microns (D₁₀); (iv) 75% or more of the total amount by volume of the drug crystals have an aspect ratio of 1-5.

In other more specific embodiments, the recrystallized columnar crystals are at least 10-500 microns in their shortest dimensions.

As used herein, laser diffraction is the methodology used for measuring and analyzing particle size and size distribution of a sample or collection of particles (e.g., the recrystallized columnar crystals and the sized crystals). More specifically, a D₁₀ value refers to the diameter at which 10% of the sample's mass is comprised of particles with a diameter less than this value. Likewise, a D₅₀ value is the diameter of the particle that 50% of a sample's mass is smaller than and 50% of a sample's mass is larger than the value.

In histogram, D₁₀, D₅₀ and D₉₀ are intercepts for 10%, 50% and 90% of the cumulative mass in the particles size. Since the density is the same for all particles, then the mass and volume are linearly related, thus the % remains the same. The method used also reports the cumulative distribution in volume.

In further more specific embodiments, the sizing step includes milling the columnar crystals in a rotor/stator homogenizer. Typically, the rotor/stator homogenizer comprises a rotating inner rotor, a stationary outer sheath, a carrier liquid medium, and a solid medium (e.g., recrystallized columnar crystals).

In yet more specific embodiments, the sizing step further comprises a step of sieving the drug crystals.

In certain embodiments, the resulting sized crystals having sizes that meet one or more of the following criteria: (i) 90% of the total mass (D₉₀) are no larger than 190 microns; (ii) 50% of the total mass (D₅₀) are less than 90±20 microns; (iii) no more than 10% of the total mass (D₁₀) are less than 30 microns; (iv) 75% or more of the total amount by volume of the bulk or collection of drug crystals have an aspect ratio of 1-3.

The process disclosed herein is particularly suitable for recrystallizing and sizing crystals of an API (i.e., drug crystals), which can be coated by a fluid bed method.

A further embodiment thus provides a collection of coated drug crystals, each being coated with a thin membrane of polymer. Suitable polymers include polyvinayl alcohol and biodegradable polyesters such as polylactic acid (PLA), polylactic-co-glycolic acid (PLGA), polycaprolactone (PCL), and poly(trimethylene carbonate) (pTMC). A preferred polymer for coating is polyvinyl alcohol. The thin membrane is about 1-10 microns thick.

The specific features are described in further detail below.

### Recrystallization

Recrystallization is a prerequisite step by first generating large, supersized crystals that can be further sized (e.g., shortened) to provide sized crystals of target dimensions. Suitably, the supersized crystals have a columnar crystal shape (also referred to as “columnar crystal”) through preferential lengthwise crystal growth. As used herein, “columnar crystals” broadly refer to crystal shapes that resemble a column, i.e., the crystals have predominant axes and transverse planes (e.g., perpendicular to the predominant axes) that are substantially the same shapes and sizes along the predominant axes.

As disclosed herein, the majority (at least 90%) of the columnar crystals have a thickness (i.e., the shortest dimension of a transverse plane perpendicular to a lengthwise axis) in the range of 50-250 microns, and an aspect ratio in the range of larger than 1 and less than 20. The columnar crystals can be as long as up to millimeters or even a centimeter long. These columnar crystals are referred to as supersized because they are both thick and elongated, which can be resized into cube-like target morphology having target dimensions in the range of 50-250 microns.

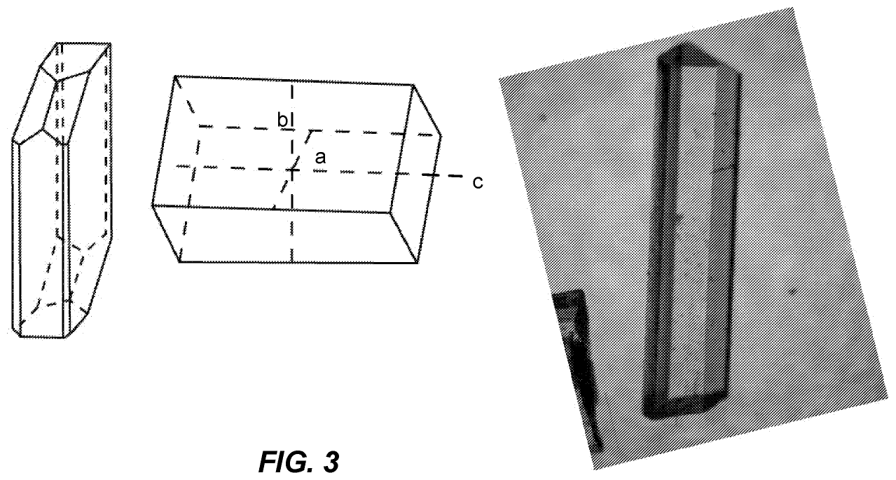

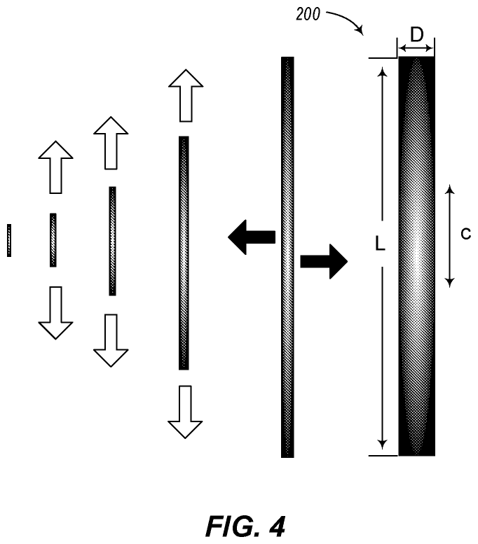

Supersized crystals are not readily achievable by conventional recrystallization due to stagnation in crystal growth. FIG. 3 shows the monoclinic Form I of fluticasone propionate (FP). The crystal structure indicates preferential crystal growth along the c axis (lengthwise) as compared to the a-b plane (transverse plane). However, this preferential growth tends to stagnate before the a-b plane can grow more than 10-15 microns in width. Indeed, known recrystallization methods, which typically involve co-solvents, anti-solvents (direct and reverse method), surface/interface modifier agents on nucleation and growth, slow and fast cooling, etc. are only capable of producing thin, needle-like crystals, in which as least one dimension fails to meet the target dimension.

In contrast to the conventional recrystallization and in accordance with an embodiment of the present disclosure, commercially available FP crystals can be recrystallized from methanol into supersized crystals. By controlling the time and condition (e.g., temperature) during recrystallization, the crystals are allowed to grow thicker along the a-b plane even after the c-axis growth slows down. FIG. 4 schematically shows the growth mechanism. The resulting supersized crystal (200) has a longest dimension (L) along the lengthwise axis and a shortest dimension (D) of the transverse plane. The aspect ratio is L/D. Suitably, the shortest dimension (D) could be substantially the same as at least one target dimension of the resized drug crystal, thus facilitating resizing by mainly breaking along the length of the supersize crystals.

In certain embodiments, the target dimensions of the recrystallized columnar drug crystals meet one or more of the following criteria: (i) 90% of the total mass are no larger than 1200 microns (D₉₀); (ii) 50% of the total mass are less than 350±180 microns (D₅₀); (iii) no more than 10% of the total mass are less than 50 microns (D₁₀); (iv) 75% or more of the total amount by volume of the drug crystals have an aspect ratio of 1-5.

### Sizing

To attain the target dimensions, the supersized crystals are suitably sized by milling in a rotor/stator homogenizer, with optional further steps of sieving and rinsing.

Conventionally, large crystals are sized via a jet or pin mill. However, they tend to generate non-uniform crystals with a large number of fines. Crystals that have c-axis epitaxial growth (e.g., FP crystals) are extremely brittle and the conventional jet or pin milling inevitably produces a very fine powder that fail to meet the target dimensions.

According to an embodiment, a rotor/stator type homogenizer is utilized, which addresses the technical limitation of the conventional resizing process. A rotor/stator homogenizer typically comprises a rotating inner rotor, a stationary outer sheath, a carrier liquid medium, and a solid medium. This type of homogenizer is conventionally used for producing fine particles or emulsions.

It has been surprisingly discovered that, by controlling the operational parameters, the supersized crystals obtained from the recrystallization step can be uniformly broken down along the lengthwise axes (e.g., c-axis), while the shortest dimension (width) is substantially unchanged. In particular, by optimizing the rotator head configuration (any combination of fine, medium, or course), rotator speed (3000-26000 rpm range), solid content (5-50% w/v of the recrystallized columnar crystals), carrier fluid types and number of runs through rotor/stator (1-10 runs), the thick, elongated crystals are gently broken down (shortened) mainly along the c-axis with minimal loss to formation of very fine particles.

The carrier fluid may include water, one or more polar organic solvents (e.g. acetone), one or more protic solvents (e.g. methanol, ethanol or isopropanol), or a combination thereof. Optionally, a surfactant may be present in the carrier fluid. Suitable surfactants include non-ionic surfactants such as polysorbates. In a more specific embodiment, polysorbate 80 (e.g., 0.1-0.5% w/v).

If needed, the sizing may further include sieving the milled (e.g., shortened) crystals to further narrow the size distribution. In various embodiments, the milled crystals may be sieved through one or more sieves to eliminate fines or remnant large crystals. Typically, two separate sieving steps are capable of producing a tight particle size distribution at scale that is also reproducible.

In addition, the sieved crystals may be rinsed to further remove fines. Typically, the sieved crystals are rinsed one or more times with a rinsing liquid comprising a surfactant, water, and optionally one or more water-miscible solvents such as methanol, ethanol, isopropanol, and the like. Suitable surfactants include polysorbates such as polysorbate 80. In certain embodiments, the surfactant is present in an amount of 0.05 to 1.0% of the rinsing liquid. For instance, a rinsing liquid may be 0.5% polysorbate 80 solution.

In certain embodiments, the sized drug crystals obtained by the process disclosed herein are a collection of drug crystals that meet one or more of the following statistical criteria: (i) 90% of the total mass (D₉₀) are no larger than 190 microns; (ii) 50% of the total mass (D₅₀) are less than 90±20 microns; (iii) no more than 10% of the total mass (D₁₀) are less than 30 microns; (iv) 75% or more of the total amount by volume of the bulk or collection of drug crystals have an aspect ratio of 1-3.

### EXAMPLES

### Example 1

### Recrystallization of Fluticasone Propionate Slow Evaporation, Isotherm at 45° C.

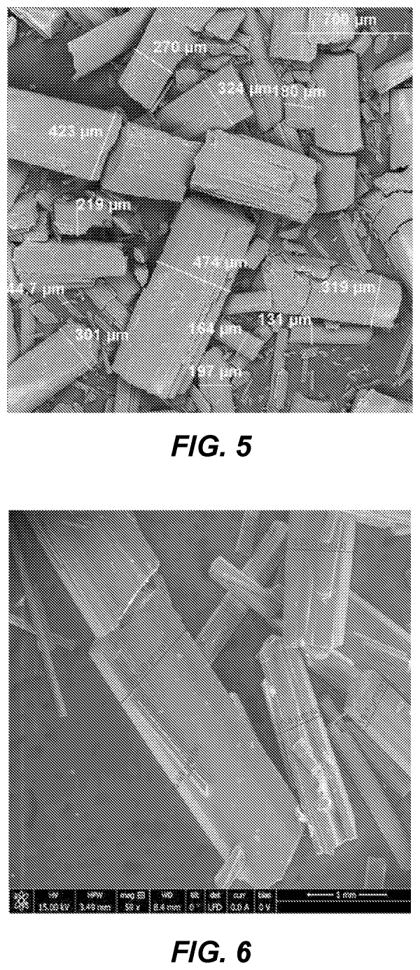

Fluticasone propionate was recrystallized from 10-15 mg/ml concentration in methanol in both small and large scales. During the slow evaporation e.g., for 72 hours, the temperature was held steadily at 45° C. The small scales were carried out in 20 mL-2 L solutions; whereas the large scales were carried out in 20-100 L. FIG. 5 shows the SEM image of the recrystallized particles in the small scale, whereas FIG. 6 shows the large-scale image. As shown, both the small and large scales produced thick elongated crystals that are suitable for further resizing.

### Example 2

### Recrystallization of Fluticasone Propionate Slow Evaporation, Isotherm at 25° C.

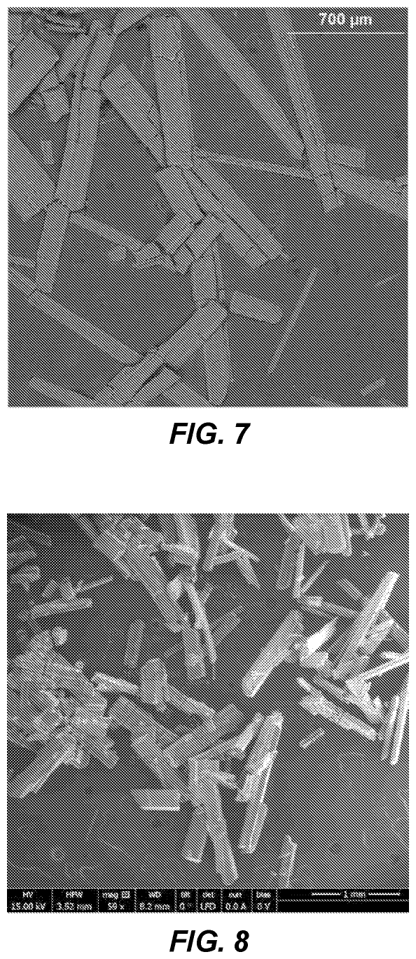

Fluticasone propionate was recrystallized from 10-15 mg/ml concentration 10-15 mg/ml concentration in methanol in both small scale of 20 mL-2 L and large scale 20-100 L. During the slow evaporation, e.g., for 20 hours, the temperature was held steadily at 25° C. FIG. 7 shows SEM image of particles produced in small-scale and FIG. 8 shows the large-scale SEM.

Different large-scale batches were run to show the reproducibility of the recrystallization method. Laser diffraction results of particle size analysis for four large scale batches are below:

| | D₁₀ | D₅₀ | D₉₀ |
|---|---|---|---|
| Batch 1 | 119 | 365 | 876 |
| Batch 2 | 97 | 323 | 838 |
| Batch 3 | 72 | 253 | 798 |
| Batch 4 | 82 | 288 | 820 |

The laser diffraction method assumes an equivalent sphere when calculating the results. The aspect ratio in the imaging analysis show a range of aspect ratio from 3 to 50, while a majority of aspect ratios are in the range of 5-20.

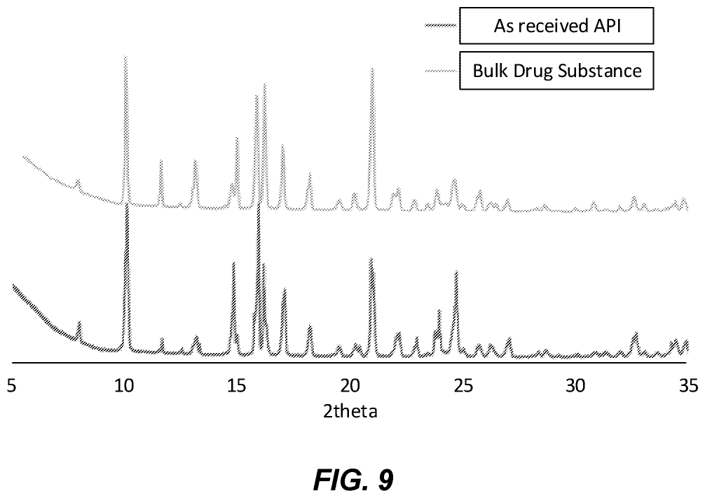

The recrystallized fluticasone propionate conforms to USP specifications, and was confirmed as the same Form I polymorphic crystal form as the original commercially available form of fluticasone propionate. FIG. 9 compares X-Ray diffraction patterns of as received (form I) and the recrystallized FP crystals. As shown, the Form I polymorph was unchanged after recrystallization.

### Example 3

### Resizing the Recrystallized Fluticasone Propionate

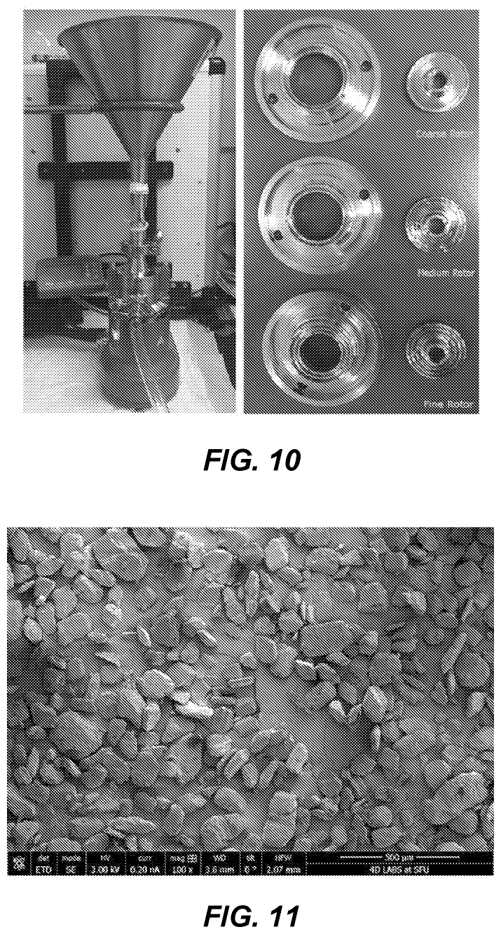

The recrystallized fluticasone propionate crystals produced by Example 1 or 2 were milled in a rotor/stator homogenizer. FIG. 10 shows a bench top model of rotor/stator homogenizer.

An exemplary set of operational parameters are as follows:
5-10% solid medium;
Carrier fluid: 0.1%-0.5% polysorbate 80 (a surfactant) in USP water
Rotor speed: 15000-20000 rpm (the mill is capable of going in range of 3000-26000 rpm)
Rotor configuration comprising coarse, medium, or fine or combination of 2 or more rotors;
Number of runs through this cycle, up to 5 times

The milled crystals were then sieved through two separate sieves to eliminate fines or remnant large crystals. FIG. 11 shows the SEM image of the resized (milling followed by sieving) drug crystals.

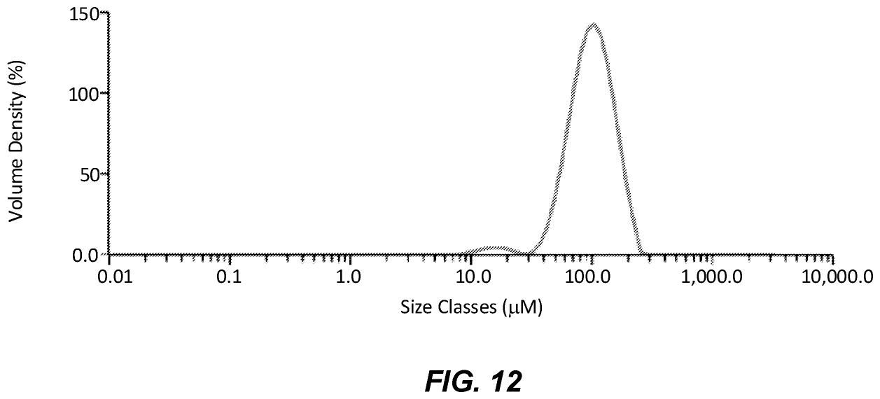

FIG. 12 shows the entire particle size distribution using the Malvern particle size analyzer. As shown, the drug crystal obtained have relatively narrow distribution and the minimal amount of fine material and absence of very fine material (no particles below 9 μm).

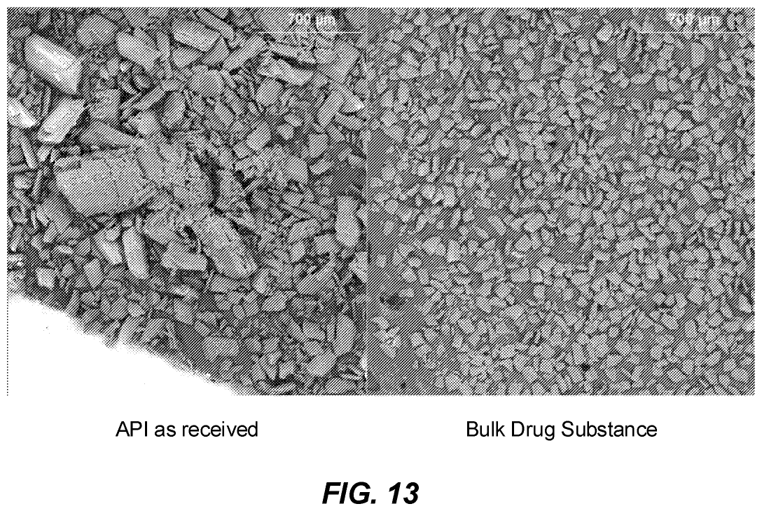

FIG. 13 shows a side-by-side comparison of the commercial source of the FP crystals showing significant variance in particle sizes and the FP drug crystals having target dimensions formed according to an embodiment of the present disclosure.

### Example 4

### Drug Release Behavior

The tight size distribution of the drug crystals improved the coating process and in turn, the release profile of the encapsulated drug. In particular, by removing the fines from the drug crystals before coating, the coating quality and efficiency were both improved. In vivo testing of the drug release behavior showed that the release from the coated particles of narrow size distribution (e.g., D₅₀ is in the range of 50-250 microns) were slower and steadier when compared to coated particles having wider size distributions

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including but not limited to [insert list], are incorporated herein by reference, in their entirety. Aspects of the embodiments can be modified, if necessary, to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

This application claims the benefit of priority to U.S. Provisional Application No. 62/832,179 filed Apr. 10, 2019, the entirety of which is incorporated by reference herein.

1. A process for providing crystals of desired size distribution and morphology, the process comprising:
providing recrystallized columnar crystals having aspect ratios of large than 1 and less than 20, the aspect ratio of a given columnar crystal being the ratio of a longest dimension along a lengthwise axis relative to a shortest dimension of a transverse plane perpendicular to the lengthwise axis; and
sizing the recrystallized columnar crystals to provide a collection of sized crystals having target dimensions, wherein the sizing includes segmenting at least a portion of the recrystallized columnar crystals along the respective lengthwise axes while retaining the dimensions of the transverse plane perpendicular to the lengthwise axis.

2. The process of claim 1 wherein the recrystallized columnar crystals further meet one or more of the following criteria:
(i) 90% of the recrystallized columnar crystals by mass are no larger than 1200 microns;
(ii) 50% of the recrystallized columnar crystals by mass are less than 350±180 microns;
(iii) no more than 10% of the recrystallized columnar crystals by mass are less than 50 microns; and
(iv) 75% or more of a total amount by volume of the recrystallized columnar crystals have an aspect ratio of 1-5.

3. The process of claim 1 wherein the recrystallized columnar crystals further meet one or more of the following criteria:
(i) 50% of the recrystallized columnar crystals by mass are in the range of 80 to 600 microns;
(ii) 75% of the recrystallized columnar crystals by mass are in the range of 50 to 800 microns; and
(ii) 90% of the recrystallized columnar crystals by mass are in the range of 20 to 1100 microns.

4. The process of claim 1 wherein the collection of the sized crystals meet one or more of the following target dimensions:
(i) 90% of the sized crystals by mass are no larger than 190 microns;
(ii) 50% of the sized crystals by mass are less than 90±20 microns;
(iii) no more than 10% the sized crystals by mass are less than 30 microns;
(iv) 75% or more of a total amount by volume of the sized crystals have an aspect ratio of 1-3.

5. The process of claim 1 wherein the collection of the sized crystals meet one or more of the following target dimensions:
(i) 50% of the sized crystals by mass are in the range of 35 to 130 microns;
(ii) 75% of the sized crystals by mass are in the range of 30 to 145 microns; and
(ii) 90% of the sized crystals by mass are in the range of 25 to 170 microns.

6. The process of claim 1 wherein the sizing comprises shortening the lengthwise axes of the recrystallized columnar crystals in a rotor/stator homogenizer to provide shortened crystals.

7. The process of claim 6 wherein the sizing further comprising sieving the shortened crystals to provide sieved crystals.

8. The process of claim 7 wherein the sieved crystals are rinsed one or more times with a rinsing liquid comprising a surfactant and one or more solvents selected from the group consisting of water, methanol, ethanol, and isopropanol.

9. The process of claim 8 wherein the rinsing liquid comprises 0.05-1% (w/w) of polysorbate 80.

10. The process of claim 1 wherein the crystals are crystals of a pharmaceutically active ingredient (API).

11. The process of claim 1 wherein the crystals are crystals of fluticasone propionate.

12. The process of claim 1 wherein the sized crystals are further individually coated with a polymer membrane by a fluid bed coating equipment.

13. The process of claim 12 wherein the polymer membrane is formed of polyvinyl alcohol, polylactic acid (PLA), polylactic-co-glycolic acid (PLGA), polycaprolactone (PCL), poly(trimethylene carbonate) (pTMC) or a combination thereof.
